(19) United States
(12) Patent Application Publication
(10) Pub. No.: US 2018/0028211 A1
(43) Pub. Date: Feb. 1, 2018

(54) ENDOSCOPIC INFLATABLE ABRADING DEVICE FOR SPINAL DISC REMOVAL

(71) Applicant: Loubert S. Suddaby, Orchard Park, NY (US)

(72) Inventor: Loubert S. Suddaby, Orchard Park, NY (US)

(21) Appl. No.: 15/221,125

(22) Filed: Jul. 27, 2016

(51) Int. Cl. A61B 17/32; A61B 17/00

(52) U.S. Cl. CPC A61B 17/32002; A61B 17/00234; A61B 2017/320024; A61B 2017/320008; A61B 2017/00685; A61B 2017/00323; A61B 17/320725

(57) ABSTRACT

In an endoscope having a working channel, an inflatable abrading device including an outer flexible sleeve, an auger arranged for controllable rotation within the sleeve, the auger arranged for rotation about a hollow shaft and a balloon having an inner surface and an outer surface, the outer surface coated with abrasive material, the balloon initially disposed within the hollow shaft and connected via a tube to a source of fluid, the balloon arranged to be extendible beyond the hollow shaft and arranged for inflation by the fluid, the tube arranged for controllable rotation.

### FIELD

The present disclosure relates generally to spinal surgery, more specifically to spinal disc removal, and, even more specifically, to an endoscopic inflatable abrading device for use in a discectomy (also known as a diskectomy).

### BACKGROUND

The spinal column, or backbone, is one of the most important parts of the body. It provides the main support, allowing us to stand upright, bend, and twist. As shown in FIG. 1, thirty three (33) individual bones interlock with each other to form the spinal column. The vertebrae are numbered and divided into regions. The cervical vertebrae (C1-C7) form the neck, support the head and neck, and allow nodding and shaking of the head. The thoracic vertebrae (T1-T12) join with the ribs to form the rib cage. The five lumbar vertebrae (L1-L5) carry most of the weight of the upper body and provide a stable center of gravity when a person moves. Five vertebrae of the sacrum S and four of the coccyx C are fused. This comprises the back wall of the pelvis. Intervertebral discs are located between each of the mobile vertebra. Intervertebral discs comprise a thick outer layer with a crisscrossing fibrous structure annulus A that surrounds a soft gel-like center, the nucleus N. Discs function like shock-absorbing springs. The annulus pulls the vertebral bodies together against the elastic resistance of the gel-filled nucleus. When we bend, the nucleus acts like a ball bearing, allowing the vertebral bodies to roll over the incompressible gel. Each disc works in concert with two facet joints, forming a spinal motion segment. The biomechanical function of each pair of facet joints is to guide and limit the movement of the spinal motion segment. The surfaces of the joint are coated with cartilage that helps each joint move smoothly. Directly behind the discs, the ring-like vertebral bodies create a vertical tunnel called the spinal canal or neuro canal. The spinal cord and spinal nerves pass through the spinal canal, which protects them from injury. The spinal cord is the major column of nerve tissue that is connected to the brain and serves as an information super-highway between the brain and the body. The nerves in the spinal cord branch off to form pairs of nerve roots that travel through the small openings between the vertebrae and the intervertebral foramens.

Various medical conditions require a surgeon to repair, remove and/or replace the aforementioned discs. For example, in one surgical procedure, known as a discectomy (or diskectomy), the surgeon removes the nucleus of the disk and replaces it with an implant. As shown in FIG. 2, it may be necessary, for example, for the surgeon to remove the nucleus of the disc between the L3 and L4 vertebrae. Disc D_{L3-L4} is shown in an enlarged view in FIG. 3. This figure also shows various anatomical structures of the spine, including facets F3A and F4A, facet joint FJ, spinous processes SP3 and SP4, transverse processes TP3A and TP44A, and intervertebral foramen IF. FIG. 4 is a top view of the section of the spinal column shown in FIG. 3, with the L3 vertebra removed to expose annulus A and nucleus N of disc D_{L3-L4}. FIG. 5 is an anterior perspective view of the section of the spinal column shown in FIG. 4. FIG. 6 is a partial cross-sectional view of the section of the spinal column shown in FIG. 5, but with vertebra L3 in place atop disc D_{L3-L4}.

One common tool used in these spinal surgical procedures is an endoscope. A representative endoscope 30 is shown in FIG. 7A. Endoscopes are complex biomedical devices. The complexity results from the need for fiberoptic bundles and multiple long narrow channels to be contained within a tubular structure that is constrained by the limited dimensions of the body cavity opening. As shown in FIG. 7A, endoscope 30 broadly comprises light guide connector 31, light guide tube 32, control body 33, and insertion tube 34. As will be described infra, the inflatable abrading device of the embodiment is introduced into the disc space via insertion tube 34. As shown in FIG. 7B, surgeon 40 uses the endoscope both to observe and guide the procedure via monitor 41, and to introduce and manipulate surgical instruments and tools during surgery on patient 45.

The endoscope is only one element of the system. Other required elements are a light source, video processor, monitor and water bottle. For the purpose of describing an endoscope in this disclosure, we refer to videoscopes, which represent a newer technology in endoscope development as compared to fiberoptic endoscopes. In videoscopes, the “viewing” fibre bundle is replaced by a miniature charged coupled device (CCD) video camera chip that transmits signals via wires.

Videoscopes include three major sections: connector 31 (sometimes referred to as the “umbilical” section), control body 33 and insertion tube 34. Endoscopes require a watertight internal compartment integrated through all components for electrical wiring and controls, which protects them from exposure to patient secretions during use and facilitates the endoscope being submerged for cleaning and subsequent disinfection. Example embodiments are not intended to be limited to any particular type of endoscope.

Control body 33 provides connections for four systems: the electrical system, the light system, the air and water system, and the suction system. A cable with video signal, light control, and remote switching from the video processor is connected in the electrical system. A watertight cap is required for leak testing and reprocessing. The electrical connector is the only opening to the internal components. The connector is inserted into the light source and directs light via the fiberoptic bundle in the light guide to the distal end of the insertion tube. Air pressure is provided from a pump to the air pipe, and the water bottle is also connected here (there is no water channel or water connection for bronchoscopes). In some endoscope models, the separate air and water channels merge just prior to the distal end where they exit through a single channel. In other models, the air and water channels are totally separate and do not merge. The air and water channels are usually of one millimeter internal diameter, which is too small for brushing. A portable or wall suction system is connected to the suction port. The Universal cord encases the electrical wiring and air, water and suction channels from the connector to the control section. Teflon® (PTFE) tubing is commonly used for channels, and advances in technology have led to more pliable and smooth materials for instrument channels with better anti-adhesion properties. The suction channel size can vary from two to 4 millimeters internal diameter depending on scope make and model. There is a biopsy port on the side of the insertion tube that allows instruments to be passed down the insertion tube to the distal end (referred to as the instrument channel or biopsy/suction channel).

Control body 33 has moveable knobs that allow the physician to control all scope functions. The angulation control knobs drive the angulation wires and control the bending section at the distal end of the insertion tube, thereby providing two-dimensional angulation. Locking mechanisms are provided to hold the bending section in a specific position. The suction cylinder and valve connects the suction channel to the instrument channel in the insertion tube. By pressing the valve button, suction can be provided to the instrument channel. The air/water cylinder and valve are similar to the suction cylinder/valve except that a two-way button valve is used in a dual channel cylinder thereby providing air or water to the lens at the distal end to wash and insufflate for better vision. Both valves are removable for cleaning. The air and water channels also require a cleaning adapter valve that is to be used at the end of each procedure. Insertion of the cleaning adapter initiates air flow through both air and water channels, and once activated, water is pumped through both channels. The instrument channel port (often referred to as the “biopsy port”) is located on the lower part of the control section. It enters the instrument channel at a Y-piece union with the suction channel. A valve is required to close the port so that suctioning may be facilitated. Remote switches present on the top of the control section are usually programmable, allowing control of the video processor (i.e., contrast, iris and image capture functions).

Most disc removal instruments have included grasping tools with alligator like jaws designed to bite and tear tissue pieces which are then withdrawn from the endoscopic working channel whereupon the disc material is removed and the instrument is reintroduced. U.S. Pat. No. 5,772,578 (Heimberger, el al.) discloses such a device which, while flexible, allows only one small piece of disc to be removed at a time. Such piece-meal removal is laborious and time consuming.

U.S. Pat. No. 8,109,957 (Stad et al.) discloses a disc nucleus removal device with cutting members in its side wall near the tip deployed by virtue of slits in the side wall sheath. Since the cutting members are actuated from the side wall of the elongate member, the blunt tip impedes the ability of the device to cut tissue directly in front of it and hence is better suited to end plate preparation than de novo disc removal. Furthermore, since the elongate member does not contain an auger, the lumen is prone to clogging, since disc fragments are well known to plug simple suction cannula because of the adhesiveness and consistency of the disc nuclear material.

Therefore, there is a long-felt need for an instrument for endoscopic disc removal which is capable of cutting and removing disc material not only on the lateral edges of the insertional axis of the device, but also on its distal most aspect where the bulk of cutting is expected to be done. Additionally, there is a long-felt need for a device not solely dependent on suction to evacuate disc material to avoid the annoying and inevitable plugging that occurs with pure single lumen suction cannula already utilized in the art. An endoscopic disc removal device that combines a retractable abrading balloon capable of cutting in 360 degrees with a controllably flexible shaft containing an auger and suction device to allow for rapid removal of disc nuclear material is needed.

### SUMMARY

According to aspects illustrated herein, there is provided a tool for an endoscope. The tool includes an auger having a hollow shaft, and an inflatable abrading device insertable through the augur hollow shaft. The auger is arranged for rotation within an outer flexible sheath, and the abrading device is similarly arranged for independent rotation by a rotating shaft within the hollow shaft. In one embodiment, the abrading device is an inflatable balloon having an inner surface and an outer surface, the outer surface coated with abrasive material. The balloon is initially disposed within the hollow shaft and connected via a tube to a source of fluid, the tube arranged for controllable rotation within the hollow shaft. In operation the balloon is moved external to the hollow shaft of the auger and arranged for rotation in a part of a body (e.g., in a disc space where it may function to morcellate tissue).

According to aspects illustrated herein, there is provided an inflatable abrading device for an endoscope having a working channel, the inflatable abrading device including: an outer flexible sheath arranged to be introduced within the working channel, a rotatable flexible member arranged for controllable rotation within the sheath, a hollow shaft arranged within the sheath, and a rotatable tube arranged to extend and retract within the hollow shaft, the rotatable tube having an inflatable abrasive member secured to a first end of said rotatable tube, the inflatable abrasive member arranged to be inflated by a source of fluid.

According to aspects illustrated herein, there is provided a method of performing a discectomy including an endoscope having a working channel, including: introducing the working channel proximate to a nucleus, removing at least a portion of the nucleus, introducing an inflatable abrading device through the working channel, the inflatable abrading device including an outer flexible sheath, an auger arranged for controllable rotation within the outer flexible sheath, the auger arranged for rotation about a hollow shaft, extending a balloon initially disposed within the hollow shaft into the nucleus, inflating the balloon, the balloon coated with abrasive material, and controllably rotating the balloon to morcellate the nucleus.

A primary object of the disclosure is to provide an inflatable abrading device for use in discectomy (diskectomy), and in other surgical procedures. It is a primary object of this disclosure to provide for an endoscopic disc removal instrument capable of being deployed down the working channel of an endoscope. It is also an object that the cutting element be both inflatable and retractable and capable of cutting directly at the tip and at the circumference of its sides. It is a further objective that the tip be steerable with simple steering wires already known in the art. Finally, it is an objective that the instrument shaft contains both a suctioning mechanism as well as a flexible auger to facilitate rapid disc tissue removal and prevent plugging known to affect present suction devices employed in disc surgery.

To achieve these objects an instrument is provided comprising an elongated central flexible core similar to a metal K-wire, around which is wound a spiral blade (flight) similar to an auger. Both the central core and spiral blade (flight) are made of metal or plastic having the ability to flex, especially near the tip.

Around the auger mechanism is a flexible sheath designed to contain and protect the auger mechanism when the mechanism is inserted or removed and especially when it is being operated by rotational force. Within the walls of the sheath are steering wires to flex the tip of the sheath similar to controllable mechanisms already known in the art and practiced in flexible cystoscopes and gastroscopes.

The central core of the auger mechanism is hollow, and allows for transport and insertion of an inflatable abrading device, i.e., a balloon coated with abrasive particles. When deployed, the balloon may be inflated to form a predetermined cutting size and shape such that cutting can occur in every direction except at the junction from the hollow shaft from which it is deployed.

When the central core is rotated, the balloon is rotated and cuts disc nuclear material into pieces which are then drawn toward the lumen of the external sheath by suction. At the opening of the lumen they encounter the spiral blade (flight) of the auger which engages them and assists in their transit along the lumen and their ultimate evacuation from the body. The central core, in essence, drives the auger and the cutting tip such that tissue morcelization and tissue evacuation can occur simultaneously with minimal risk of obstruction of the central lumen. Suctioning force is applied as well through the auger channel within the endoscope.

These, and other objects and advantages will be readily appreciable from the following description of preferred embodiments of the invention and from the accompanying drawings and claims.

### BRIEF DESCRIPTION OF THE SEVERAL VIEWS OF THE DRAWINGS

The nature and mode of operation of the present disclosure will now be more fully described in the following detailed description of the embodiments taken with the accompanying figures, in which:

FIG. 1 is an anterior perspective view of spinal column 10;

FIG. 2 is an anterior perspective view of the lumbar section of spinal column 10;

FIG. 3 is a lateral perspective view of L3, L4 vertebrae and disc D_{L3-L4} and related spinal anatomy;

FIG. 4 is a top view of a section of the spinal column, taken generally along line 4-4 in FIG. 3;

FIG. 5 is an enlarged anterior perspective view of the spinal column shown in FIG. 2, except with vertebra L3 and all other structure above L3 removed;

FIG. 6 is a partial cross-sectional view of the L4 vertebra and D_{L3-L4} disc taken generally along line 6-6 in FIG. 5, and also shows L3 in cross-section;

FIG. 7A is a view of a typical endoscope;

FIG. 7B illustrates use of the endoscope shown in FIG. 7A by a surgeon performing a discectomy (diskectomy);

FIG. 8 illustrates a preliminary step in a discectomy (diskectomy) procedure, namely, introduction of a needle to pierce the annulus and nucleus of a disc;

FIG. 9 illustrates another preliminary step in a discectomy (diskectomy) procedure, namely, introduction of a first dilator into the disc;

FIG. 10 illustrates a further preliminary step in a discectomy (diskectomy) procedure, namely, introduction of a larger dilator into the disc;

FIG. 11 illustrates the working tube of the endoscope proximate the disc just prior to introduction of surgical instruments into the disc;

FIG. 12 illustrates introduction of a rongeur into the nucleus of the disc;

FIG. 13 is a view similar to that of FIG. 12 showing introduction of the inflatable abrading device (prior to inflation) of the present disclosure into the disc space, with line 14-14 representing the cross-sectional view depicted in FIG. 14;

FIG. 14 is a cross-sectional view of working channel 35, taken generally along line 14-14 in FIG. 13, showing the endoscope and auger tube 70;

FIG. 15 is a view similar to that of FIG. 13, except showing the inflatable device in an inflated state and rotating to morcellate the disc material for removal by the auger;

FIG. 16A is a perspective view of the auger tube of the device;

FIG. 16B is a view similar to that of FIG. 16A, but showing part of the auger tube cut away to expose the auger of the device;

FIG. 16C is a view similar to that of FIG. 16B, but showing part of the spiral blade (flight) of the auger in phantom to show the hollow auger shaft of the device;

FIG. 16D is a perspective view similar to that of FIG. 16A, showing the inflatable abrading device extending outwardly from the hollow auger shaft of the device;

FIG. 17 is a phantom perspective view of a part of the working tube of an endoscope, showing placement of the auger tube and the endoscope tube;

FIG. 18 is a fragmentary front perspective view of the auger tube and inflatable abrading device;

FIG. 19 is a fragmentary rear perspective view of the auger tube and inflatable abrading device shown in FIG. 18;

FIG. 20 is an enlarged side view of the inflatable abrading device shown in FIG. 18;

FIG. 21 is an enlarged front view of the inflatable abrading device shown in FIG. 18;

FIG. 22 is a cross-sectional view of the inflatable abrading device, taken generally along line 22-22 in FIG. 20;

FIG. 23 is a cross-sectional view similar to the view in FIG. 22 but illustrating an alternative embodiment of the inflatable abrading device;

FIG. 24 is a fragmentary front perspective view of the auger tube and an alternative embodiment of the inflatable abrading device; and,

FIG. 25 is a cross-sectional view of the alternate inflatable abrading device of the embodiment, taken generally along line 25-25 in FIG. 24.

### DETAILED DESCRIPTION OF EMBODIMENTS

At the outset, it should be appreciated that like drawing numbers on different. drawing views identify identical, or functionally similar, structural elements of the invention. While the present invention is described with respect to what is presently considered to be the preferred aspects, it is to be understood that the invention as claimed is not limited to the disclosed aspect. The present invention is intended to include various modifications and equivalent arrangements within the spirit and scope of the appended claims.

The term “balloon” as used in the present disclosure is intended to mean any inflatable member which can be elastomeric or non-elastomeric and made of any material.

Furthermore, it is understood that this invention is not limited to the particular methodology, materials and modifications described and, as such, may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

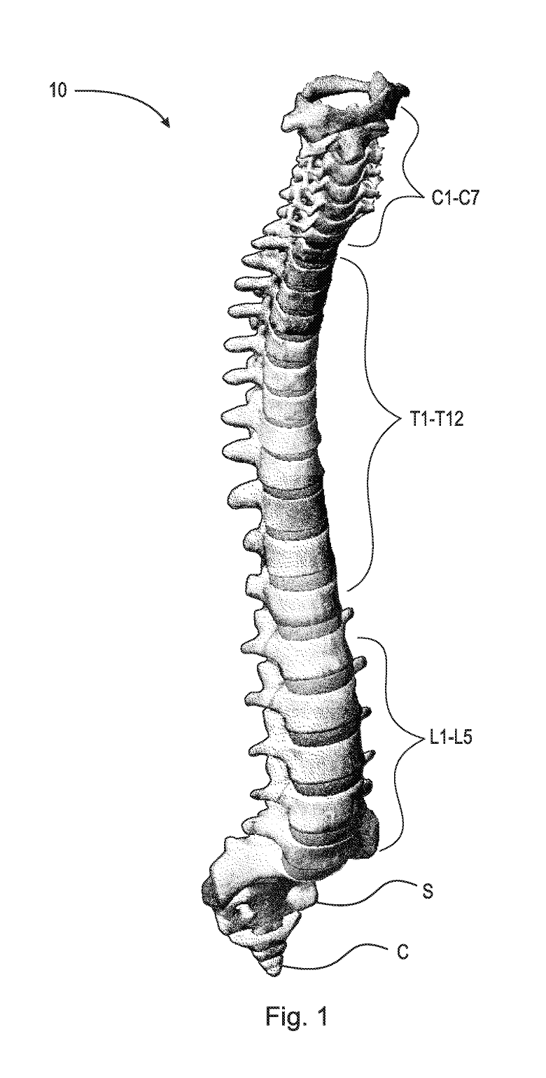

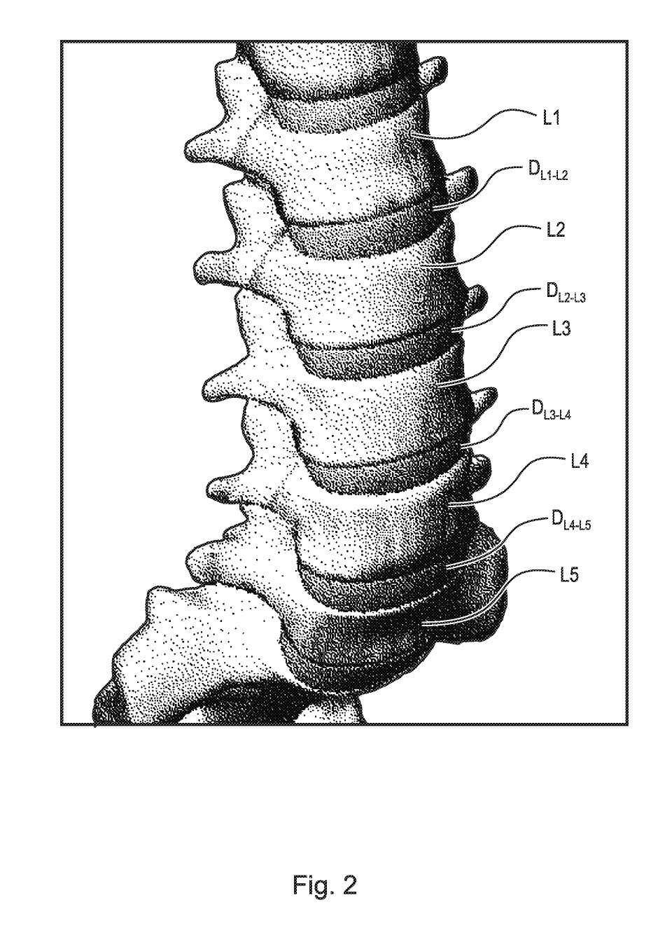

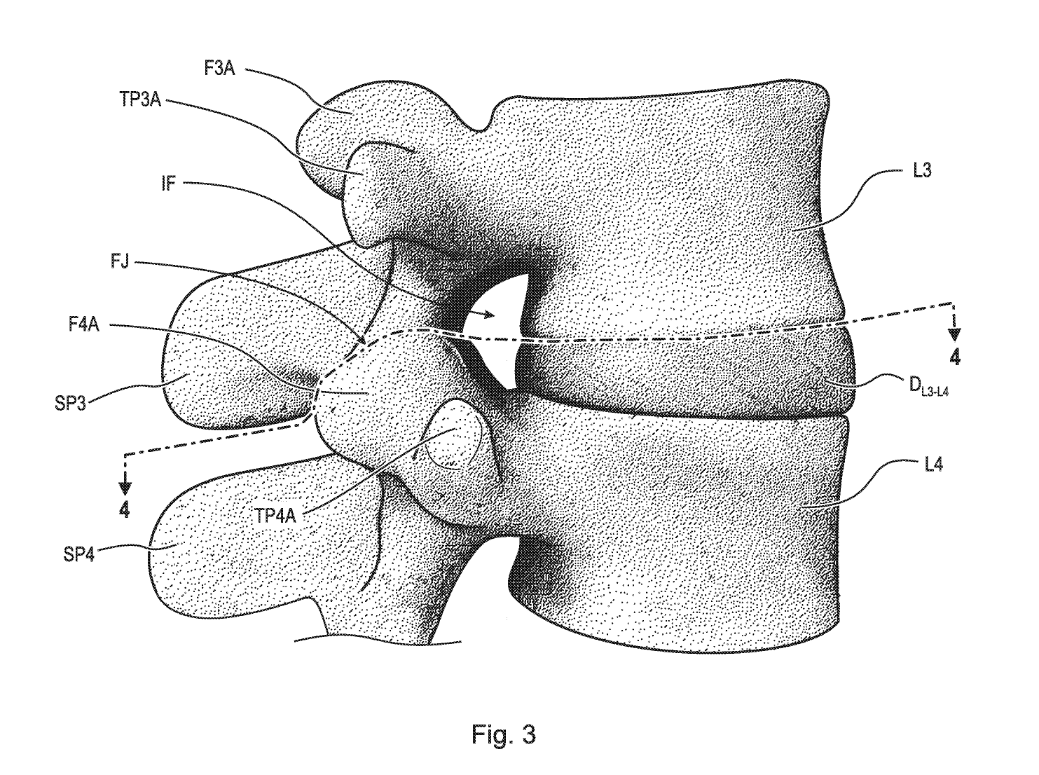

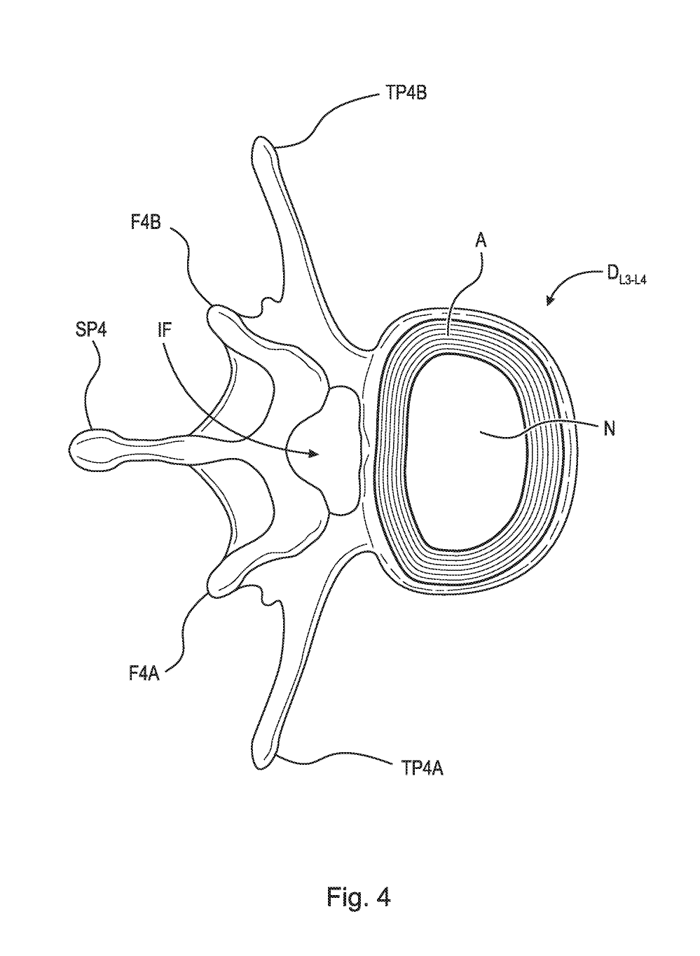

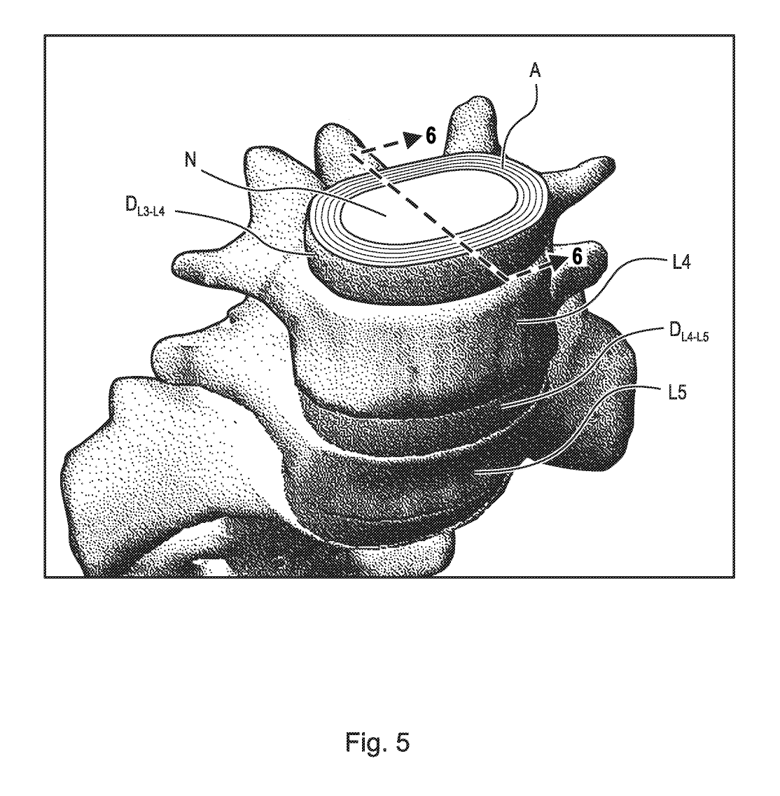

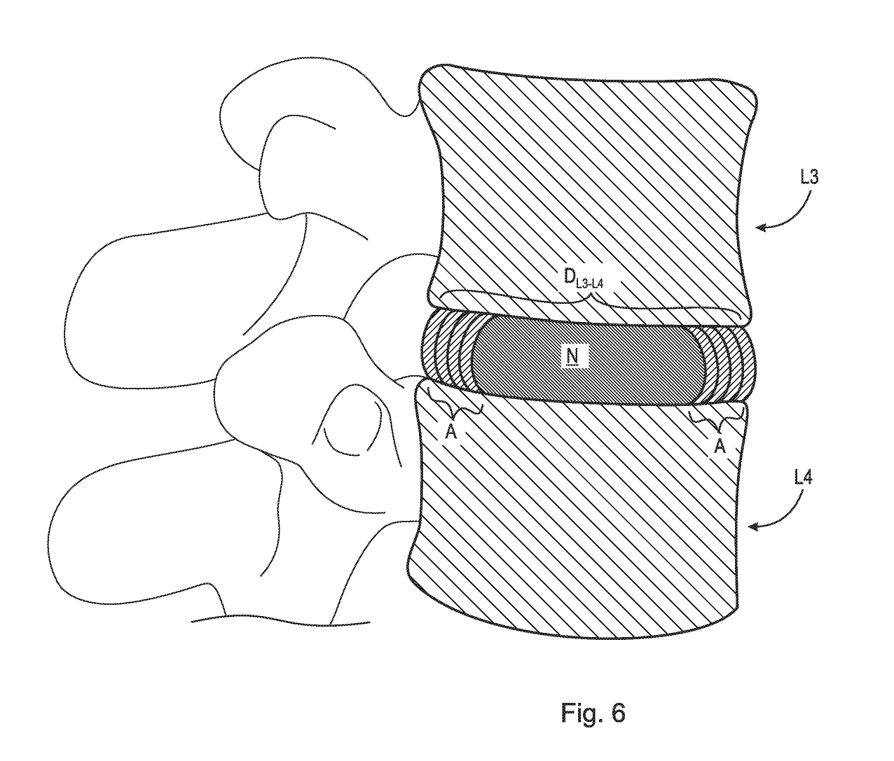

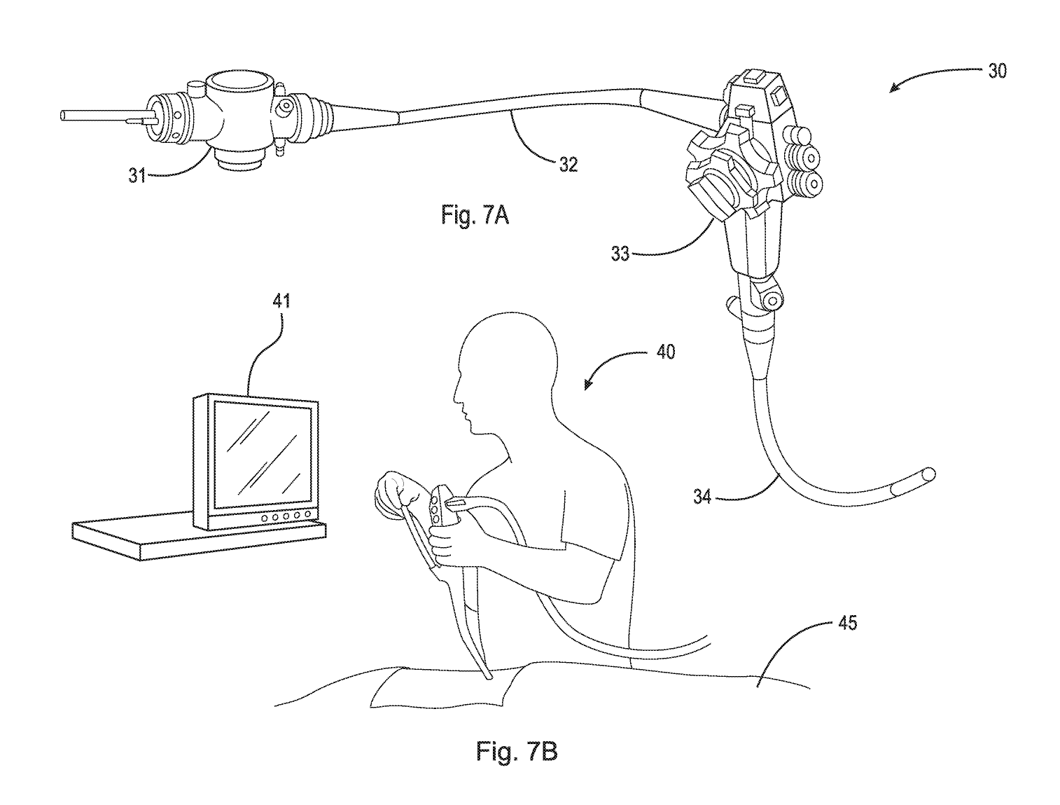

Adverting now to the Figures, and as described previously, FIGS. 1-6 depict various parts and sections of spinal anatomy, and FIGS. 7A and 7B depict a typical endoscope for use by a surgeon on a patient. We now focus on the discectomy (diskectomy) procedure itself, first with reference to FIG. 8, which shows first dilator 50, having shaft portion 51 and point portion 52 piercing annulus A and nucleus N of disc D_{L3-L4}.

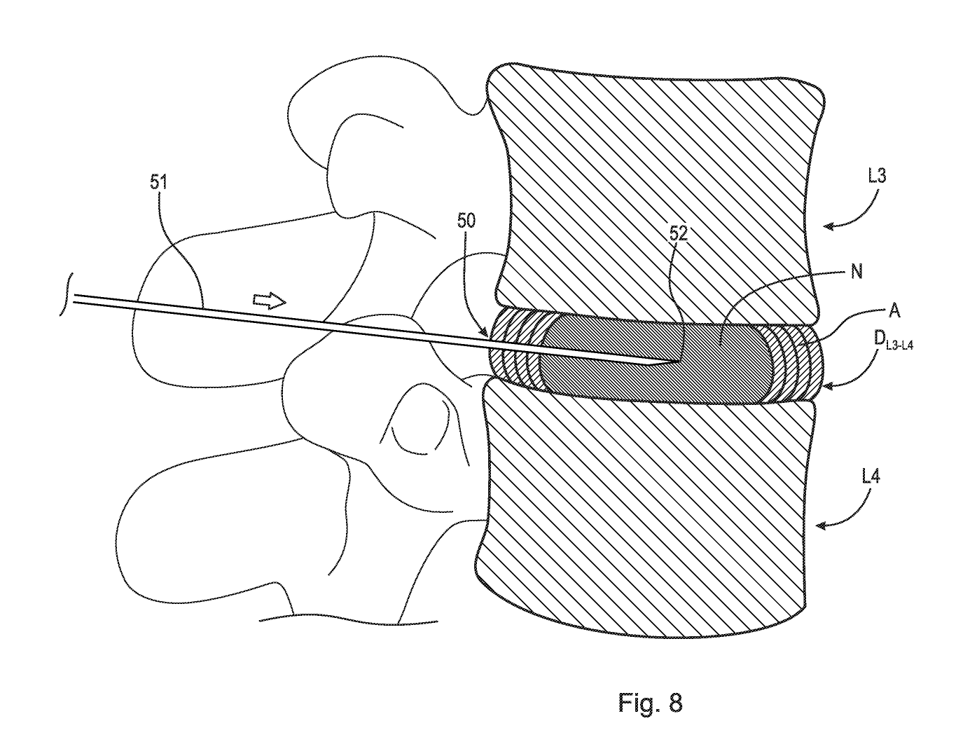

Once the surgeon has pierced the annulus and entered disc D_{L3-L4} with dilator 50, he enlarges the entry channel with a plurality of increasingly larger dilators, as best shown in FIG. 9. This figure shows dilators 53 and 54 being introduced down shaft portion 51 (shown in FIG. 8), and eventually into annulus A and the center of nucleus N of disc D_{L3-L4}.

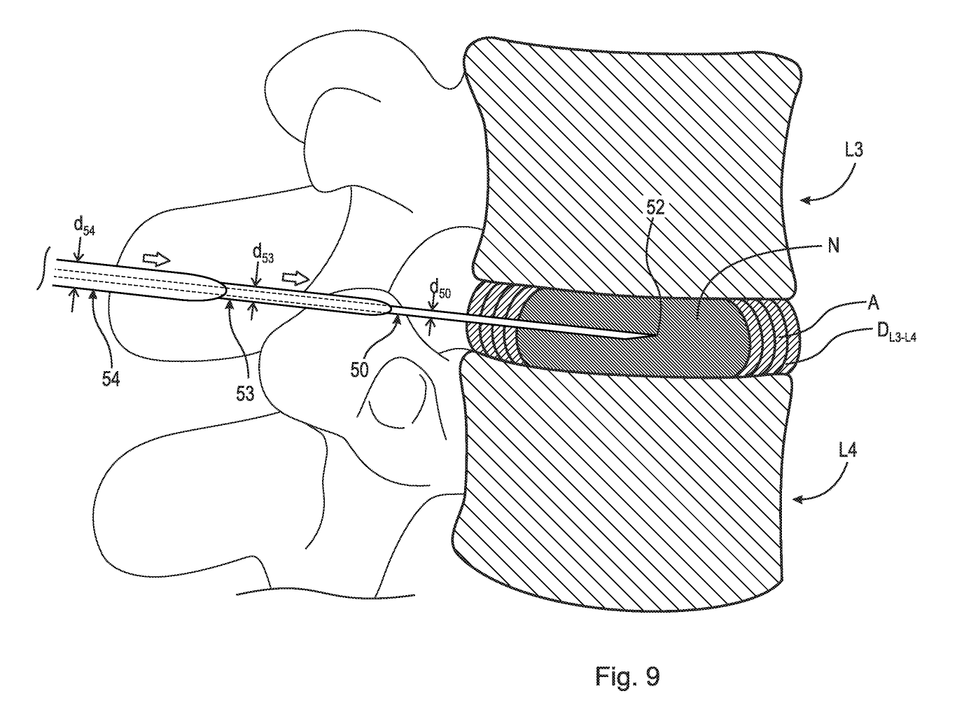

FIG. 10 illustrates a further preliminary step in a discectomy (diskectomy) procedure, namely, introduction of larger dilators 53 and 54 into nucleus N of disc D_{L3-L4} along shaft portion 51. Dilator 55 is introduced as well to further dilate nucleus N. This is done to increase the size of the opening in annulus A and the size of disc cavity 58 for the eventual introduction of insertion tube 34 (shown in FIG. 11) proximate to nucleus N. Once the dilators have made the opening large enough, insertion tube 34 is introduced over the dilators.

FIG. 11 illustrates introduction of insertion tube 34 with endoscopic working channel 35 of the endoscope through annulus A of disc D_{L3-L4} proximate to nucleus N just prior to introduction of surgical instruments into disc cavity 58. When insertion tube 34 is in place proximate to nucleus N, all dilators are removed.

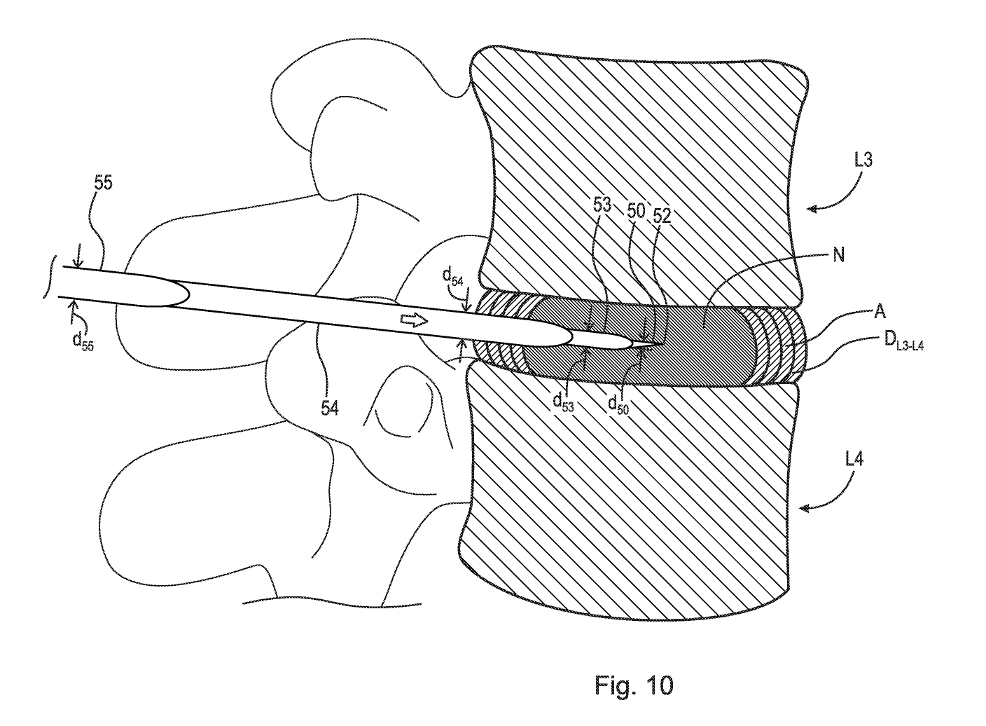

FIG. 12 illustrates introduction of rongeur 57 through endoscopic working channel 35 of insertion tube 34 into disc cavity 58 in nucleus N of disc D_{L3-L4}. Rongeur 57 is used to remove portions of nucleus N to enlarge disc cavity 58 for introduction of inflatable abrading device 60. Endoscope tube 76 transmits a video feed to monitor 41 to assist physician 40.

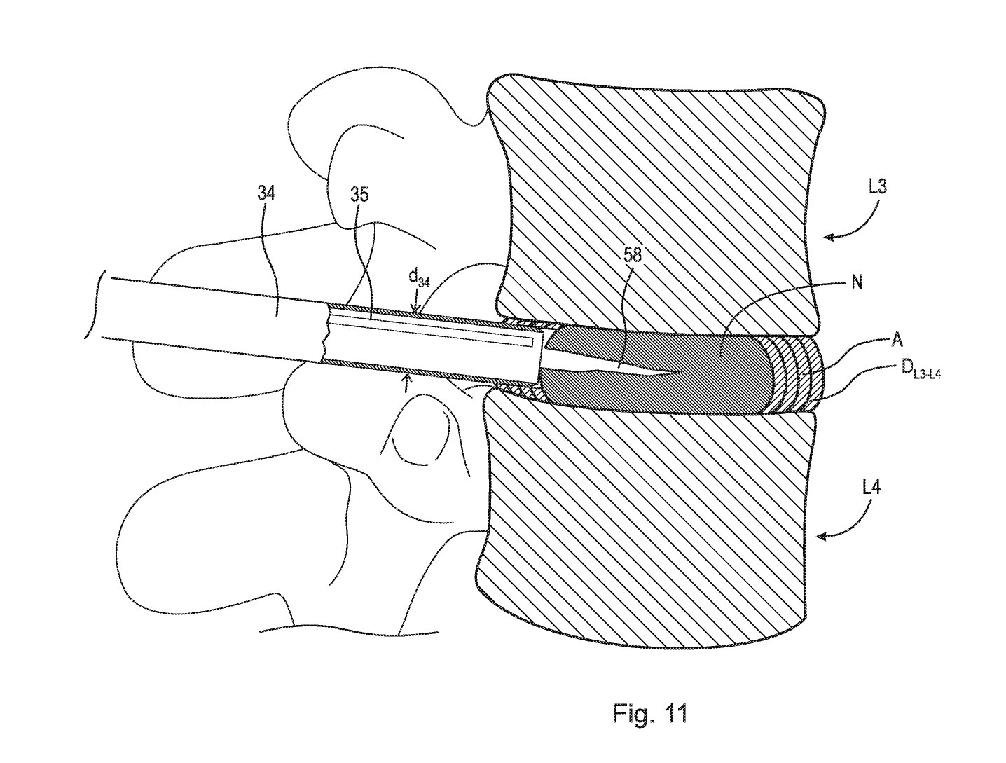

FIG. 13 illustrates the introduction of inflatable abrading device 60 or balloon (prior to inflation) through auger channel 74. Inflatable abrading device 60 is made of a material with a one to nine micron thick outer surface 62 covered with stainless steel abrasive particles 63 adhered to inflatable abrading device surface 62. Device can also be made of a para-aramid synthetic fiber, such as Kevlar brand para-aramid synthetic fiber, polytetrafluoroethylene (PTFE), polyether ether ketone (PEEK), fabric, or other hard plastic or metal which is inexpensive so that it is disposable, or biodegradable material. Particles 63 can also be pre-formed or molded within device 60. Device 60 can be elastomeric or non-elastomeric. Particles 63 can also be made of steel, titanium, diamond, or other abrasive metal. Inflatable abrading device 60 is positioned in disc cavity 58. Auger tube 70 is introduced through endoscopic working channel 35. Auger 71 and spiral blade (flight) 72 are not yet rotating in auger tube 70. Both auger shaft 73 and spiral blade (flight) 72 are made of metal or plastic having the ability to flex, especially near the tip.

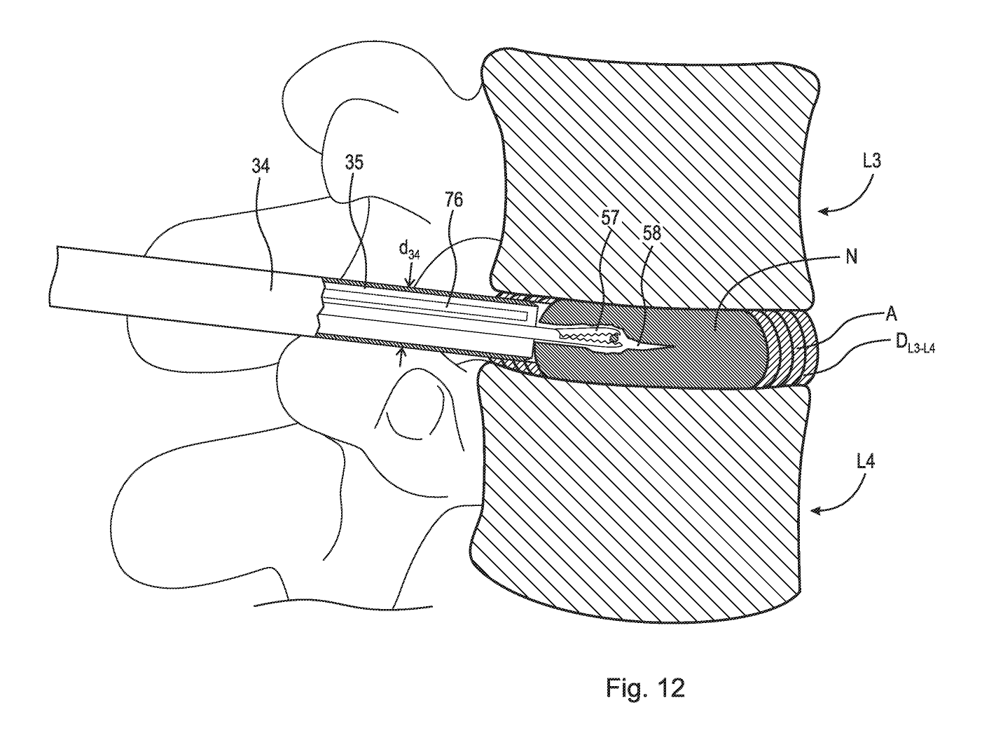

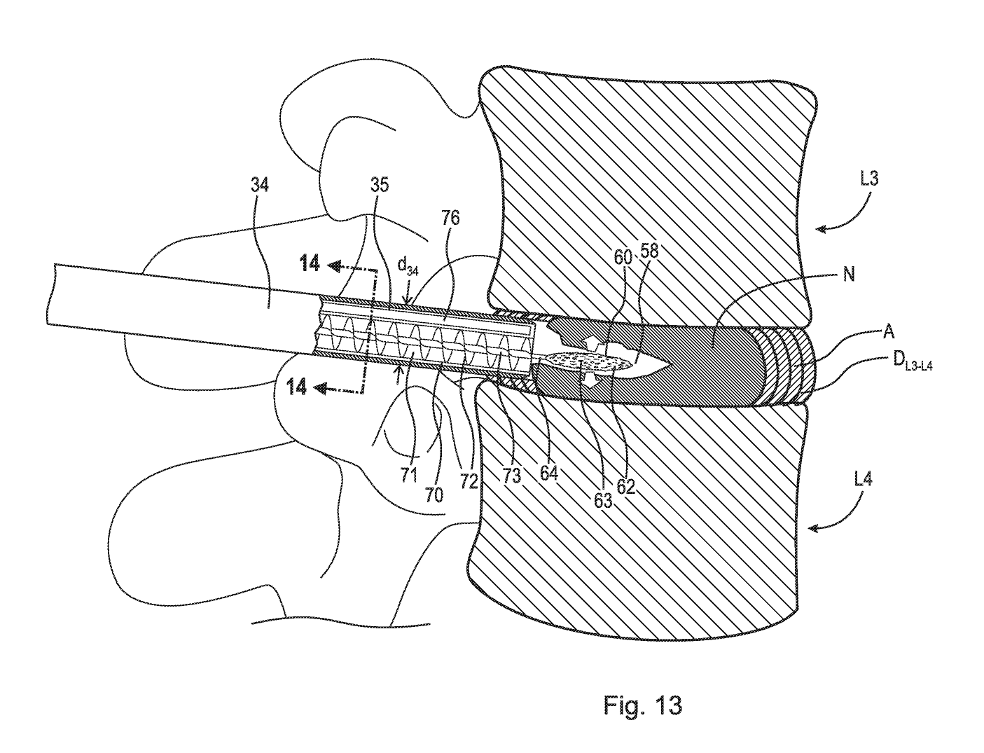

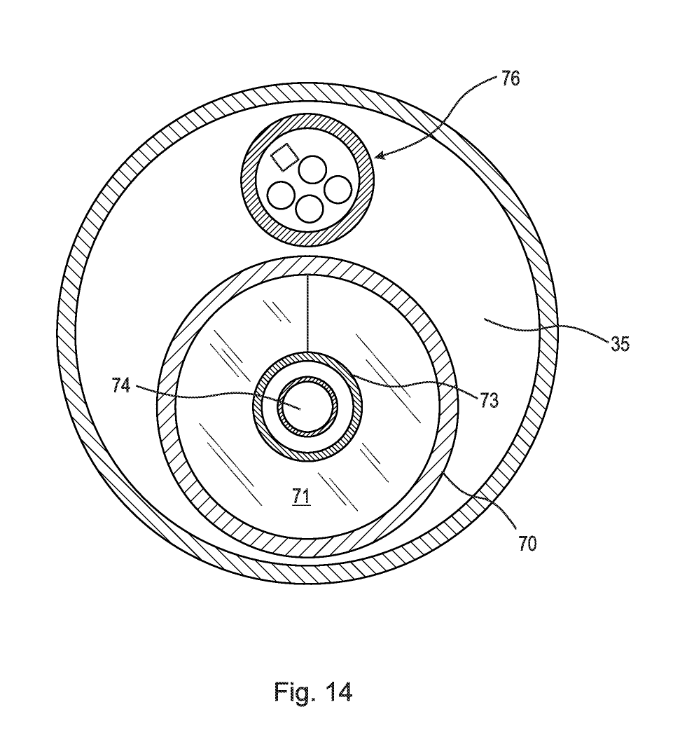

FIG. 14 is a cross-sectional view taken generally along line 14-14 in FIG. 13, illustrating the inside of insertion tube 34 and endoscopic working channel 35. Endoscope tube 76 is proximate to auger tube 70. Auger tube 70 contains auger 71 coiled around auger shaft 73. Inflatable abrading device 60 (not shown in FIG. 14) is introduced through auger channel 74.

FIG. 15 depicts the present disclosure performing a discectomy (diskectomy). Inflatable abrading device 60 is connected to rotatable tube 64, through which inflatable abrading device 60 is initially inflated with saline or Conray® media for fluoroscopic visualization, for example. The media is introduced into inflatable abrading device 60 by a hydraulic pump, for example. Once the media has inflated inflatable abrading device 60 to the desired level, the inflation is maintained and inflatable abrading device 60 is rotated to morcellate disc D_{L3-L4}. While disc D_{L3-L4} is being morcellated, saline is introduced into the disc space, and disc material 61 is evacuated through auger tube 70 by rotating auger 71 and applying a suctioning force from the other end of auger tube 70. Once disk D_{L3-L4} is morcellated, media may be removed from inflatable abrading device 60 by a hydraulic pump, for example. Inflatable abrading device 60 and auger 71 may rotate in the same or in different directions, and each can reach a maximum speed of approximately three hundred surface feet per minute, for example. In practice, physician 40 is able to adjust the speed at which both inflatable abrading device 60 and auger 71 rotate. In the preferred embodiment, inflatable abrading device 60 rotates at a faster rate than auger 71.

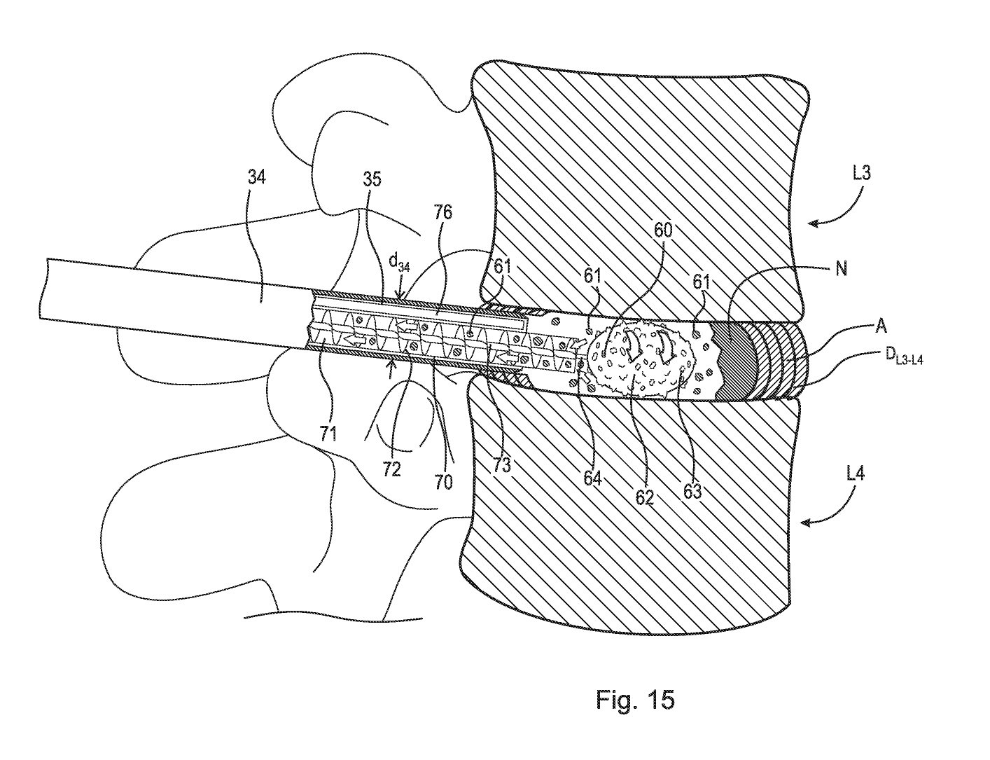

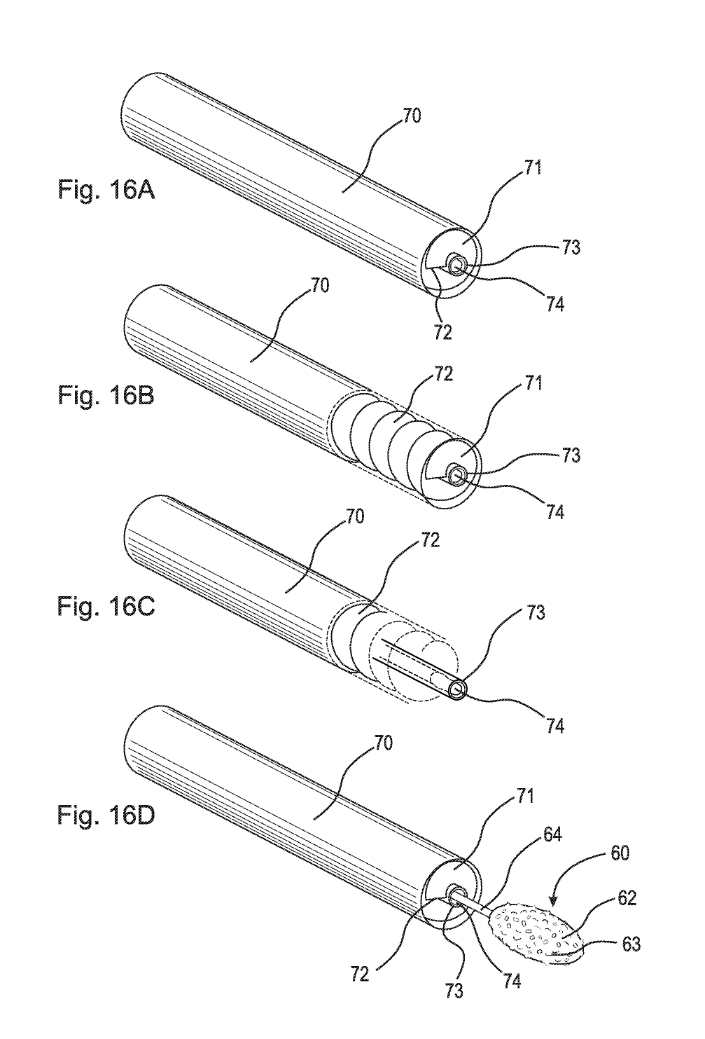

FIG. 16A is a perspective view of auger tube 70 of the device, with auger 71 partially exposed. FIG. 16B is a view similar to that of FIG. 16A, but showing a portion of auger tube 70 in phantom to expose auger 71 and spiral blade (flight) 72. FIG. 16C is a view similar to that of FIG. 16B, but showing part of spiral blade (flight) 72 in phantom to expose the hollow auger shaft 73 of the device. Inflatable abrading device 60 is introduced through auger channel 74. FIG. 16D is a perspective view similar to that of FIG. 16A, showing inflatable abrading device 60 inflated and attached to rotatable tube 64 and extending outwardly from auger shaft 73 of the device. It should be understood that although a particular auger and blade are illustrated, other auger and blade configurations are contemplated. For example, spiral blade 72 can include apertures or protrusions. Alternatively, spiral blade 72 can include a tapering blade or a non-continuous blade or a fan-like component. Auger 71 can include multiple blades as well.

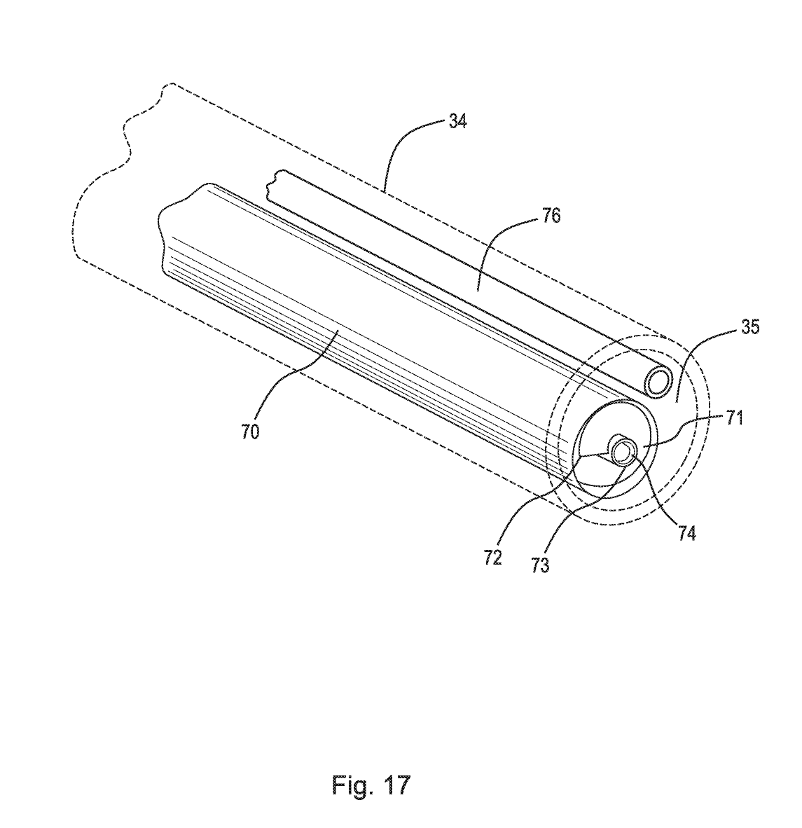

FIG. 17 is a phantom perspective view of insertion tube 34 of the endoscope, showing the placement of auger tube 70 and endoscope tube 76. Endoscope tube 76 transmits video to monitor 41 to assist physician 40. Auger channel 74 is concentrically arranged within auger tube 70. Device 60 connected to rotatable tube 64 is arranged to extend through auger channel 74.

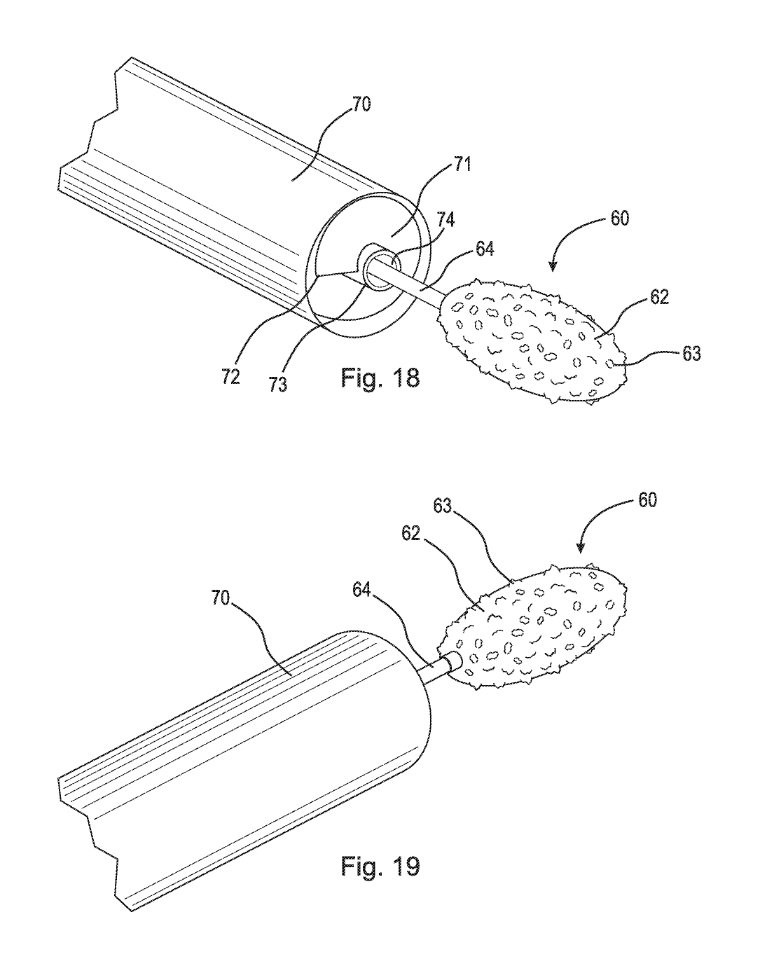

FIG. 18 is a fragmentary front perspective view of auger tube 70 and inflatable abrading device 60 of the embodiment, similar to the one depicted in FIG. 16D.

FIG. 19 is a fragmentary rear perspective view of auger tube 70 and inflatable abrading device 60 of the embodiment. Device 60 is connected to rotatable tube 64 by an o-ring for example, or any tonic joint or mechanical gasket. In an example embodiment, rotatable tube 64 includes a groove for the o-ring or an alternative.

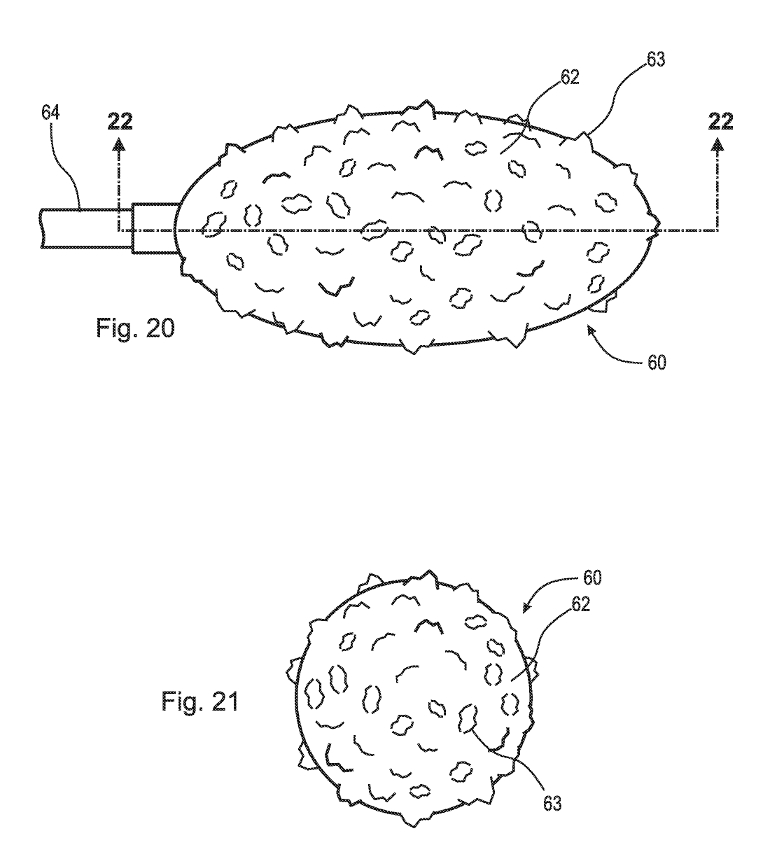

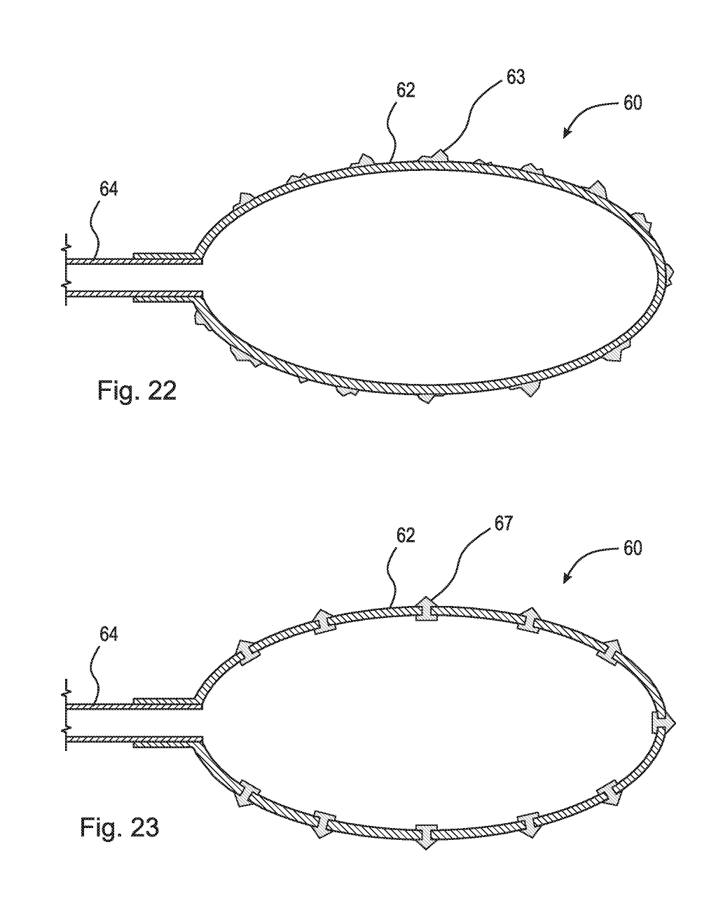

FIG. 20 is an enlarged side view of inflated inflatable abrading device 60 in an inflated state, attached to rotatable tube 64 with inflatable abrading device surface 62 and abrasive particles 63. Line 22-22 represents the cross-section of inflatable abrading device 60 presented in FIG. 22.

FIG. 21 is a front view of inflatable abrading device 60, with inflatable abrading device surface 62 and abrasive particles 63.

FIG. 22 is a cross-sectional view of the image depicted in FIG. 20. This shows the inside of the inflated inflatable abrading device 60. Media is introduced into inflatable abrading device 60 through the opening at the end of rotatable tube 64. Abrasive particles 63 on inflatable abrading device 64 in this embodiment are adhered or formed on inflatable abrading device surface 62.

FIG. 23 is similar to FIG. 22. However, FIG. 23 shows an alternative embodiment of the abrading particles, alternative abrading particles 67. Abrading particles 67 are stainless steel, or equivalent, studs that are inserted through the inside of inflatable abrading device 60, and protrude through inflatable abrading device surface 62. Particles 67 can also be adhered on inflatable abrading device surface 62.

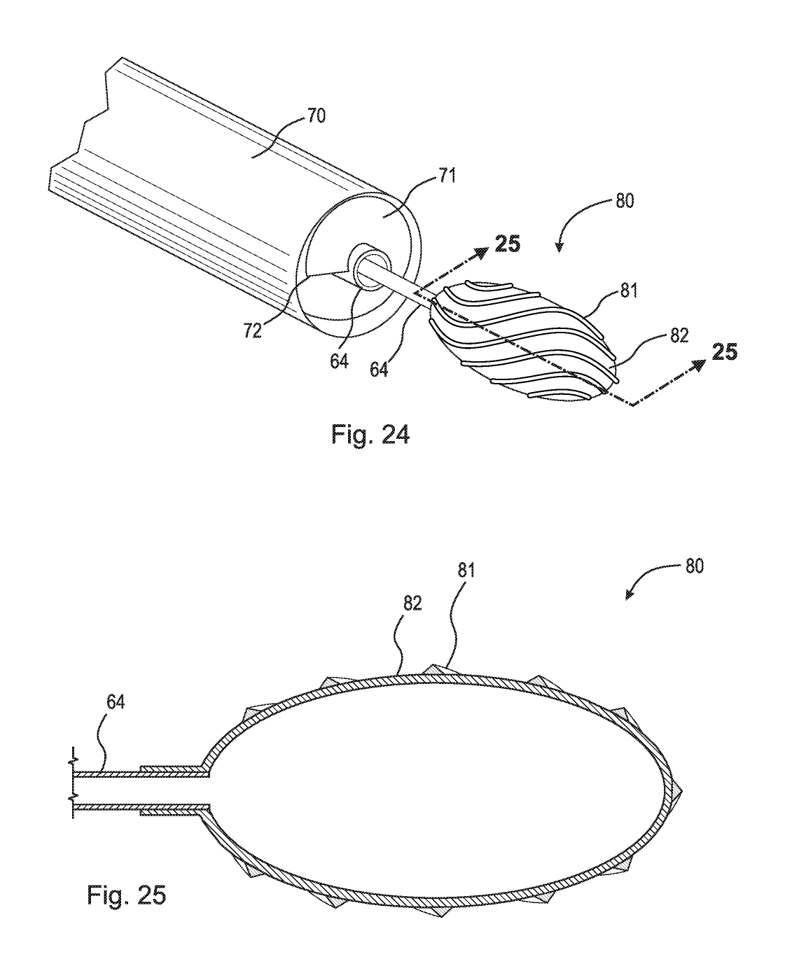

FIG. 24 is similar to FIG. 18. However, FIG. 24 depicts alternative inflatable abrading device 80, with alternative abrading vanes 82. All properties of alternative inflatable abrading device 80 are the same as inflatable abrading device 60, except for the parallel helical line pattern of alternative abrading vanes 82. Although a particular configuration of vanes are illustrated, other configurations of vanes may be used. The vanes can also be more or less pronounced.

FIG. 25 is similar to FIGS. 22 and 23. However, FIG. 25 is taken generally along line 25-25 in FIG. 24, and depicts alternative inflatable abrading device 80, with alternative abrading particles 81 adhered or otherwise affixed onto alternative abrading device surface 82. Alternative inflatable abrading device 80 is also attached to rotatable tube 64.

Thus it is seen that the objects of the invention are efficiently obtained, although changes and modifications to the invention should be readily apparent to those having ordinary skill in the art, which changes would not depart from the spirit and scope of the invention as claimed.

### LIST OF REFERENCE NUMBERS

10 Spinal column
C1-C7 Cervical vertebrae
T1-T9 Thoracic vertebrae
L1-L5 Lumbar vertebrae
S Sacrum
C Coccyx
D_{L1-L2} Disc
D_{L2-L3} Disc
D_{L3-L4} Disc
D_{L4-L5} Disc
F Facet
FJ Facet joint
SP Spinous process
TP Transverse process
IF Intervertebral foramen
A Annulus
N Nucleus
DH Disc height
30 Endoscope
31 Light guide connector
32 Light guide tube
33 Control body
34 Insertion tube
d₃₄ Diameter of Insertion tube
35 Endoscopic working channel
40 Surgeon
41 Monitor
45 Patient
50 Dilator
51 Shaft portion
52 Point portion
53 Dilator
54 Dilator
55 Dilator
d₅₀ Diameter of dilator
d₅₃ Diameter of dilator
d₅₄ Diameter of dilator
57 Rongeur
58 Disc cavity
60 Inflatable abrading device
61 Disc particle
62 Inflatable abrading device surface
63 Abrasive particles
64 Rotatable tube
66 Alternative abrasive particles
70 Auger tube
71 Auger
72 Spiral blade (flight)
73 Auger shaft
74 Auger channel
76 Endoscope tube
80 Alternative inflatable abrading device
81 Alternative inflatable abrading device surface
82 Alternative abrasive vanes

I claim:

1. In an endoscope having a working channel, an inflatable abrading device, comprising:
an outer flexible sheath;
an auger arranged for controllable rotation within said outer flexible sheath, said auger arranged for rotation about a hollow shaft; and,
a balloon having an inner surface and an outer surface, said outer surface coated with abrasive material, said balloon initially disposed within said hollow shaft and connected via a tube to a source of fluid, said balloon arranged to be extendible beyond said hollow shaft and arranged for inflation by said fluid, said tube arranged for controllable rotation.

2. The inflatable abrading device recited in claim 1, wherein said outer flexible sheath is arranged for insertion within the working channel.

3. The inflatable abrading device recited in claim 1, wherein said balloon is operatively arranged for morcellation of a disc.

4. The inflatable abrading device recited in claim 1, further comprising a suctioning means to remove morcellated disc material from an intervertebral space.

5. The inflatable abrading device recited in claim 1, further comprising at least one steering wire within said outer flexible sheath operatively arranged to flex and steer said auger.

6. The inflatable abrading device recited in claim 1, wherein said balloon is arranged for precise and variable inflation by a surgeon dependent upon the nature of a surgical procedure and a patient's spinal anatomy.

7. The inflatable abrading device recited in claim 1, wherein said auger is flexible.

8. The inflatable abrading device recited in claim 1, wherein said auger includes a single continuous spiral flight.

9. The inflatable abrading device recited in claim 1, wherein said abrasive material includes at least one blade.

10. An inflatable abrading device for an endoscope having a working channel, said inflatable abrading device comprising:
an outer flexible sheath arranged to be introduced within the working channel;
a rotatable flexible member arranged for controllable rotation within said sheath;
a hollow shaft arranged within said sheath; and,
a rotatable tube arranged to extend and retract within said hollow shaft, said rotatable tube having an inflatable abrasive member secured to a first end of said rotatable tube, said inflatable abrasive member arranged to be inflated by a source of fluid.

11. The inflatable abrading device recited in claim 10, wherein said rotatable flexible member is an auger.

12. The inflatable abrading device recited in claim 10, wherein said rotatable tube and said inflatable abrasive member are integral.

13. The inflatable abrading device recited in claim 10, wherein said outer flexible sheath, said rotatable flexible member and said hollow shaft are concentric.

14. The inflatable abrading device recited in claim 10, further comprising a suctioning means proximate said rotatable flexible member.

15. The inflatable abrading device recited in claim 10, wherein said inflatable abrasive member is arranged to morcellate a disc.

16. The inflatable abrading device recited in claim 10, further comprising at least one steering wire within said outer flexible sheath operatively arranged to flex and steer said auger.

17. The inflatable abrading device recited in claim 10, wherein said rotatable flexible member includes a single continuous flight.

18. The inflatable abrading device recited in claim 10, wherein said inflatable abrasive member is arranged to be deflated after a disc has been morcellated and removed from an intervertebral space.

19. The inflatable abrading device recited in claim 10, wherein said rotatable tube is arranged to rotate faster than said rotatable flexible member within said working channel.

20. A method of performing a discectomy including an endoscope having a working channel, comprising:
introducing said working channel proximate to a nucleus;
removing at least a portion of said nucleus;
introducing an inflatable abrading device through said working channel, said inflatable abrading device including an outer flexible sheath, an auger arranged for controllable rotation within said outer flexible sheath, said auger arranged for rotation about a hollow shaft;
extending a balloon initially disposed within said hollow shaft into said nucleus;
inflating said balloon, said balloon coated with abrasive material; and,
controllably rotating said balloon to morcellate said nucleus.
